(19) United States
(12) Patent Application Publication
(10) Pub. No.: US 2001/0007255 A1
(43) Pub. Date: Jul. 12, 2001

(54) Test head

(76) Inventor: Willi Stumpf, Schriesheim (DE)

Correspondence Address: VOLPE AND KOENIG, P.C., SUITE 400, ONE PENN CENTER, 1617 JOHN F. KENNEDY BOULEVARD, PHILADELPHIA, PA, 19103, US

(21) Appl. No.: 09/757,004

(22) Filed: Jan. 9, 2001

(30) Foreign Application Priority Data
Jan. 11, 2000 (DE) 200 00 379.8

(51) Int. Cl. A62B 7/00; A62B 9/00; A61M 15/00; A61M 16/00; A62B 18/00

(52) U.S. Cl. 128/200.24

(57) ABSTRACT

Test head for respirator and diving masks with a shape approximating the human head and having at least one air duct traversing the interior of the test head. The test head includes an opening, preferably in the area of the mouth, to which one end of the air duct is attached while the other end can be connected to air supply or measuring equipment. The air duct includes a stopper element for selective pressure-tight closing of the air duct in an area of the test head.

### BACKGROUND

The present invention concerns a test head for respirator and diving masks with a shape approximating the human head and with at least one air duct traversing the interior of the test head, the test head including an opening, preferably in the area of the mouth, to which is attached one end of the air duct while the other end can be connected to air supply or measuring equipment.

Such test heads, as they are known, for example, from DE-U 296 05 844, are employed for the purpose of checking the tightness of respirator masks and metered breathing valves (so-called lung machines). For this purpose, the air duct runs from the mouth area to the base of the test head, via which the masks and lung machines can be flushed by an artificial lung or via which the flow of air produced by a blower can be guided through the masks and lung machines. Since the breathing volume and the air flow need to be of a certain quantity and since the test head should itself exhibit the minimum possible internal resistance, the cross-section of the air duct needs to be correspondingly large.

In testing tightness, a vacuum or over pressure of about 5-15 mbars is produced in the mask or in the lung machine, and the change in the pressure differential is determined over a certain period of time. For this purpose, however, it is necessary to keep the volume of the air duct as small as possible with respect to the volume of the mask or the lung machine in order to avoid obtaining any distortion of test values as a result of the additional volume of the air duct. Because the leakage determined during tightness testing is based on the total volume of mask and air duct, the leakage rate is naturally smaller than if it had been related only to the mask volume.

Especially in the case of lung machines, which because of their construction exhibit a small intrinsic volume, an excessive dead-space volume of the air duct would have a clearly negative effect.

With this in mind, the present invention is based on the object of making available a test head of the type mentioned above which satisfies both of the contradictory requirements described and makes possible, on the one hand, large air throughput, but, on the other hand, does not significantly adversely affect the measured results through an excessively large volume of the air duct.

### SUMMARY

This object is solved according to the present invention through the fact that the air duct running in the area of the test head includes a stopper element for selective pressure-tight closing of the air duct. This results in the advantage that even with a still large cross-section of the air duct, the volume of the air duct having an effect on the measurement of tightness can be substantially reduced by positioning the stopper element near one end of the air duct, i.e., near the mouth region. Through such an arrangement, the remainder of the air duct between stopper element and supply or measuring equipment is partitioned off, and the relevant volume cannot adversely affect tightness measurements.

It is especially advantageous if the stopper element is designed to be reversibly expandable and closes the air duct in the activated state and frees at least a substantial cross-section of the air duct in the deactivated state. Such an expandable stopper element occupies relatively little room in the air duct in the deactivated state and thus only slightly obstructs the air duct. However, on the other hand, it can be sufficiently expanded in the activated state so that the entire cross-section of the air duct is sealed off.

This expandable stopper element appropriately is formed of an inflatable balloon body which can be activated especially pneumatically. Precisely compressed air is very suitable as the activating medium in the present case of application since no special supply lines are required here—such as would be the case with water, for example.

The stopper element is appropriately positioned in the air duct and, in the activated state, acts against the inner wall of the air duct. As a result, sealing problems and other complications can be managed. In addition, the stopper element is appropriately stored in the deactivated state in a container positioned in the air duct and expands beyond the container upon activation. In this way, one can ensure that any adverse effect on the passage of air is not especially great in the case of a deactivated stopper element.

As concerns the deactivation of the stopper element, this can occur through release of the inflating medium, i.e., especially compressed air, and/or through mechanical withdrawal back into the storage container.

If measuring lines are positioned in the air duct, it is recommended that the balloon body functioning as the stopper element be adapted thereto, i.e., that the body, for example, have an annular shape or exist as a two-chamber balloon body consisting of two balloon bodies arranged in parallel.

### BRIEF DESCRIPTION OF THE DRAWINGS

Further features and advantages of the present invention become clear from the following description of the preferred embodiments on the basis of the drawings. In the drawings:

FIG. 1 is a cross-sectional view of the test head with a deactivated stopper element;

FIG. 2 is a view similar to FIG. 1 of the test head with an activated stopper element;

FIG. 3 is a cross-sectional view of a test head with an alternative deactivated stopper element; and

FIG. 4 is a view of the test head from FIG. 3 with an activated stopper element.

### DETAILED DESCRIPTION OF THE PREFERRED EMBODIMENTS

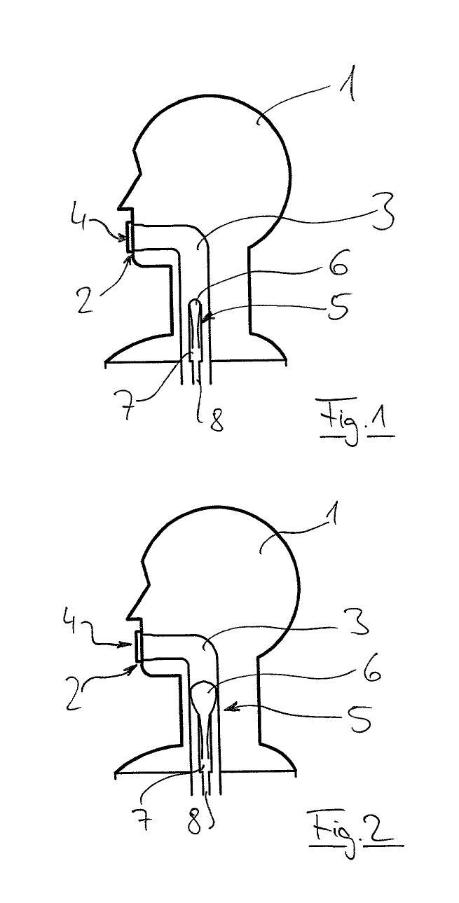

FIG. 1 shows a test head 1 having a shape similar to the human head and serving in testing the tightness as well as the performance behavior of respirator masks and lung machines. These are positioned in the area of the mouth 2 of the test head and are supplied with air via an air duct 3. The air duct extends through the test head from an opening 4 in the area of the mouth of the test head to an air-supply or measuring devices (not shown).

It can be readily seen already from FIG. 1 that the volume of the air duct 3 is not negligible and would distort values measured in checks of tightness since any leakage rate would be calculated on the basis of a completely false starting volume and not approximately from the volume of the respiratory mask or the lung machine. The present invention intervenes here and provides the air duct with a stopper element 5 which includes a reversibly expandable balloon body 6 which is stored in a container 7 in the deactivated state (see FIG. 1). In the activated state, however, the balloon body 6 is inflated via an air duct 8 by compressed air or gas and is forced out of the container, where it expands until it contacts the inner wall of the air duct 3 and completely seals off the cross-section of the air duct. As a result, one obtains a dead-space volume, i.e., a volume of the air duct between the opening 4 and the stopper element 6 which is substantially reduced compared to the dead-space volume of the mask and the air duct without the stopper element. Through suitable selection of the position of the stopper element in the air duct, the dead-space volume can also be appropriately adjusted.

Once the desired tightness measurement has been completed, the balloon body can be deactivated again, for which purpose the air is evacuated from the balloon body and the balloon body, because of its elasticity, draws itself back into the storage container.

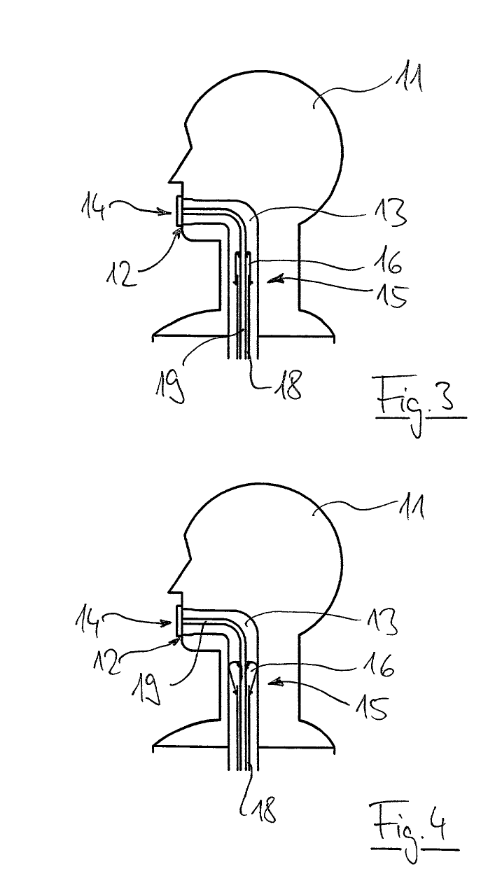

The embodiment shown in FIGS. 3 and 4 differs in the fact that a measuring duct 19 is positioned in the air duct, which excludes the use of a balloon body corresponding to FIGS. 1 and 2. Instead, a stopper element 15 with an annular balloon body 16 is positioned in air duct 13, with the balloon body 16 surrounding the measuring duct at its periphery and being inflatable with compressed air via an air duct 18, whereupon the balloon body 16 comes to rest in its activated state against the inner wall of the air duct 13. In addition, the stopper element 15 is designed without a storage container, with the balloon body 16 becoming secured pressure-tight with its annular inner surface against the measuring duct and with its annular outer surface against the likewise annular air duct 18.

In summary, a correct result is attained during tightness measurements through the present invention without substantially reducing the flow through the air duct. This is made possible as a result of the stopper element of the present invention, which element is designed and activated in extremely simple fashion and through which automated testing also remains possible due to the fact that the stopper element can be activated solely through pneumatic inflation and deactivated through subsequent evacuation.

What is claimed is:

1. Test head for at least one of respirator and diving masks having a shape approximating a human head, comprising at least one air duct (3,13) traversing an interior of the test head, the test head (1,11) including an opening (4,14), to which a first end of the air duct is attached while a second end of the air duct is adapted to be connected to air supply or measuring equipment, the air duct includes a stopper element (5,6,15,16) for selective pressure-tight closing of the air duct in an area of the test head.

2. Test head according to claim 1, wherein the stopper element (5,6,15,16) is designed to be reversibly expandable and closes the air duct (3,13) in an activated state and frees at least a substantial cross-section of the air duct in a deactivated state.

3. Test head according to at least claim 2, wherein the stopper element (5,6,15,16) is comprised of an inflatable balloon body (6,16).

4. Test head according to claim 2, wherein the stopper element (5,6,15,16) is pneumatically activated.

5. Test head according to claim 1, wherein the stopper element (5,6,15,16) is positioned in the air duct (3,13).

6. Test head according to claim 2, wherein the stopper element (5,6,15,16), in the activated state, acts against an inner wall of the air duct (3,13).

7. Test head according to claim 2, wherein the stopper element (5,6) is stored in the deactivated state in a container (7) positioned in the air duct (3) and the stopper element expands beyond the container upon activation.

8. Test head according to claim 2, wherein the deactivation of the stopper element (5,6,15,16) occurs through release of the inflating medium and/or through mechanical withdrawal back into a storage container (7).
